(19) United States
(12) Patent Application Publication
(10) Pub. No.: US 2019/0270822 A1
(43) Pub. Date: Sep. 5, 2019

(54) ANTIBODY-CONJUGATED NANOPARTICLES AND MEDICAL USES THEREOF

(71) Applicant: Trustees of Tufts College, Medford, MA (US)

(72) Inventors: Sandra L. Ayres, Sudbury, MA (US); Qiaobing Xu, Somerville, MA (US); Kristy L. Meadows, North Grafton, MA (US)

(21) Appl. No.: 16/320,029

(22) PCT Filed: Jul. 11, 2017
(86) PCT No.: PCT/US17/41491

§ 371 Date: Jan. 23, 2019

Related U.S. Application Data
(60) Provisional application No. 62/366,826, filed on Jul. 26, 2016.

(51) Int. Cl. C07K 16/28; A61P 35/00; A61K 38/17; A61K 47/54; A61K 31/351; A61K 31/337; A61K 9/51

(52) U.S. Cl. CPC C07K 16/2869; A61P 35/00; A61K 38/1796; A61K 9/51; A61K 31/351; A61K 31/337; A61K 47/543

(57) ABSTRACT

An antibody-conjugated nanoparticle, 50 to 1000 nm in size, containing an anti-Mullerian hormone receptor II (AMHRII) antibody that is conjugated to a nanocomplex formed of a lipid-based delivery agent and a cytotoxin, the delivery agent and the cytotoxin being non-covalently bonded to each other. Also disclosed are a method of preparing such an antibody-conjugated nanoparticle and use thereof for inducing sterilization in a subject and for treating an AMHRII-associated condition.

### BACKGROUND

As a medical technique, sterilization is widely used to control overpopulation. Currently, the most effective sterilization method for pets involves surgical removal of the pets' ovaries or testes.

Surgical sterilization, requiring medical equipment and extensive training, is an expensive endeavor. Further, it can result in post-surgery morbidity.

Non-surgical sterilization, e.g., use of a zinc gluconate solution, is convenient, but requires analgesia or anesthesia, which can lead to post-operation morbidity as well.

There is a need for a new method that does not have the above drawbacks for inducing sterilization.

### SUMMARY

To meet this need, disclosed herein is a method of using an antibody-conjugated nanoparticle for inducing sterilization of a subject, such as a cat and a dog. The method includes two steps: (1) identifying a subject in need of sterilization and (2) administering to the subject an effective amount of an antibody-conjugated nanoparticle. The antibody-conjugated nanoparticle, 50 to 1000 nm in size, contains an anti-Mullerian hormone receptor II (AMHRII) antibody and a nanocomplex formed of a lipid-based delivery agent and a cytotoxin that are non-covalently bonded to each other. The cytotoxin, upon delivery into gonad cells, suppresses the formation or development of sperm or ova, thereby inducing sterilization of the subject.

Also within the scope of this invention is an antibody-conjugated nanoparticle that can be used in the sterilization method described above.

To reiterate, the antibody-conjugated nanoparticle contains an AMHRII antibody and a nanocomplex formed of a lipid-based delivery agent and a cytotoxin.

The lipid-based delivery agent typically contains a cationic lipid, which can be formed from a primary or secondary amine and an electrophile, e.g., an epoxide, an acrylate, or an acrylamide. The cationic lipid can contain a disulfide bond and be bioreducible in the presence of a cysteine residue, e.g., glutathione.

The cytotoxin can be an unmodified natural protein, e.g., RNase A and saporin, or a natural protein modified with a chemical moiety, e.g., RNase A-Aco and saporin-Aco (Aco being a chemical moiety derived from aconitic anhydride). Note that the cytotoxin can also be a small molecule having a molecular weight of 900 Daltons or less, e.g. pacilitaxel and doxorubicin.

In one embodiment, the cytotoxin is a natural protein modified with a chemical moiety. The chemical moiety generally contains an anionic group, a pH responsive group, a disulfide group, a hydrophobic group, a light responsive group, a reactive oxygen species responsive group, or a combination thereof. The chemical moiety can be linked to the natural protein via an amide group, an ester group, an ether group, a thioether group, a disulfide group, a hydrazone group, a sulfenate ester group, an amidine group, a urea group, a carbamate group, an imidoester group, or a carbonate group. Preferably, the chemical moiety contains an anionic group, a pH responsive group, or a disulfide group; and is linked to the natural protein via an amide group, an ester group, a disulfide group, a thioester group, or a carbamate group.

The lipid-based delivery agent can be bonded to the cytotoxin via an electrostatic interaction or a hydrophobic interaction.

The antibody-conjugated nanoparticle can be used for inducing sterilization by targeting the AMHRII expressed in gonad cells, e.g., granulosa cells, sertoli cells, theca cells, and leydig cells. It can also be employed in targeting the AMHRII expressed in other cells, e.g., cancer cells, for treating an AMHRII-associated condition.

Thus, also covered by this invention is a method of using the antibody-conjugated nanoparticle described above for treating an AMHRII-associated condition in a subject. The method includes two steps: (1) identifying a subject that has an AMHRII-associated condition and (2) administering to the subject in need thereof an effective amount of an antibody-conjugated nanoparticle. The antibody-conjugated nanoparticle delivers the cytotoxin contained therein into cells expressing AMHRII, thereby killing the cells.

Examples of the AMHRII-associated condition include, but are not limited to, prostate cancer, breast cancer, endometrial cancer, cervical cancer, ovarian cancer, polycystic ovarian disease, and menopause.

Finally, this invention further covers a method of preparing the antibody-conjugated nanoparticle described above. The method includes four steps, namely, (i) providing a synthetic lipid formed from an electrophile and a primary or secondary amine, the electrophile being an epoxide, an acrylate, or an acrylamide; (ii) mixing the synthetic lipid and a cytotoxin to form a nanocomplex; (iii) mixing the nanocomplex with a lipid material to obtain a lipid-modified nanocomplex; and (iv) conjugating the lipid-modified nanocomplex with AMHRII antibody to form an antibody-conjugated nanoparticle that contains the cytotoxin.

The details of the invention are set forth in the description below. Other features, objects, and advantages of the invention will be apparent from the following detailed description of several embodiments, and also from the appending claims.

### DETAILED DESCRIPTION

Disclosed first in detail herein is a method of using an antibody-conjugated nanoparticle for inducing sterilization in a subject.

According to the American Society for the Prevention of Cruelty to Animals, it is estimated that there are over 70 million stray dogs and cats in the United States. The development of a non-surgical method of sterilization has the potential to decrease pet overpopulation by reducing the mortality, cost, and inconvenience associated with surgical sterilization. The method of using an antibody-conjugated nanoparticle for inducing sterilization is accomplished by targeting the anti-Mullerian hormone receptor II (AMHRII), which is expressed primarily in gonads.

Certain types of gonad cells, such as the granulosa cells, sertoli cells, theca cells, and leydig cells, support the formation or development of the sperm and ova by controlling their meiosis. Without these gonad cells, the sperm and ova cannot develop or simply die. On the other hand, these gonad cells produce hormones that impact the hypothalamic-pituitary-gonadal axis (which refers to the hypothalamus, pituitary gland, and gonadal gland) responsible for the cycling and estrous behavior in females, and feedback for testosterone production and male reproductive behaviors in males. Upon administering into a subject, the antibody-conjugated nanoparticle, which contains an AMHRII antibody and a nanocomplex formed of a lipid-based delivery agent and a cytotoxin, delivers the cytotoxin contained therein into gonad cells, causing these cells to undergo apoptosis, and suppresses the formation or development of sperm or ova, thereby inducing sterilization in the subject.

Advantages of this method over existing methods for inducing sterilization include, but are not limited to, (1) it works in both males and females of potentially all mammalian species, (2) this method involves a single injection of an antibody-conjugated nanoparticle into a subject, in which the nanoparticle can be administered as vaccines to a pet animal or administered via rabies poles or dart guns to a feral animal, and (3) no analgesia or anesthesia is needed and no medical training is required to perform the method.

Further disclosed in detail herein is an antibody-conjugated nanoparticle, which can be used in the above described method for inducing sterilization.

To reiterate, the antibody-conjugated nanoparticle contains an AMHRII antibody and a nanocomplex formed of a lipid-based delivery agent and a cytotoxin, the delivery agent and the cytotoxin being non-covalently bonded to each other, in which the AMHRII antibody is conjugated to the nanocomplex to form a nanoparticle that contains the cytotoxin, the nanoparticle having a size of 50 to 1000 nm.

The lipid-based delivery agent typically contains a cationic lipid, which can be formed from an electrophile and a primary or secondary amine, in which the electrophile is an epoxide, an acrylate, or an acrylamide.

Each of the epoxide, acrylate, and acrylamide can contain a C₁-C₂₀ alkyl or C₁-C₂₀ heteroalkyl group. Examples of the epoxide, acrylate, and acrylamide include, but are not limited to,

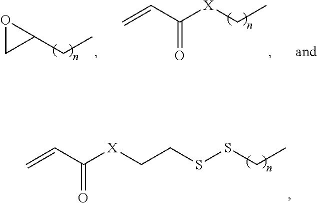

in which X is O or NH and n is 9-17.

Examples of the primary or secondary amine include, but are not limited to,

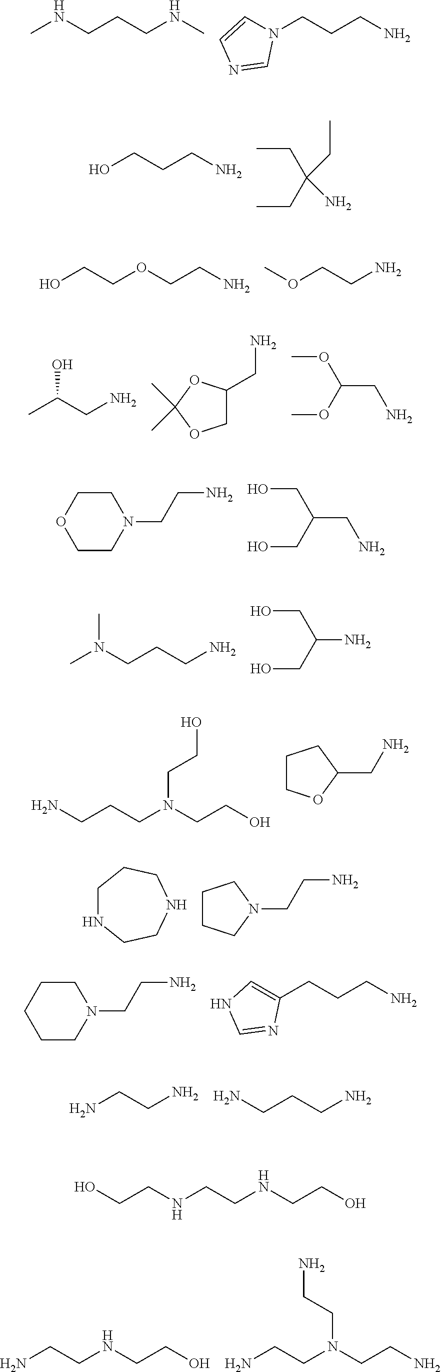

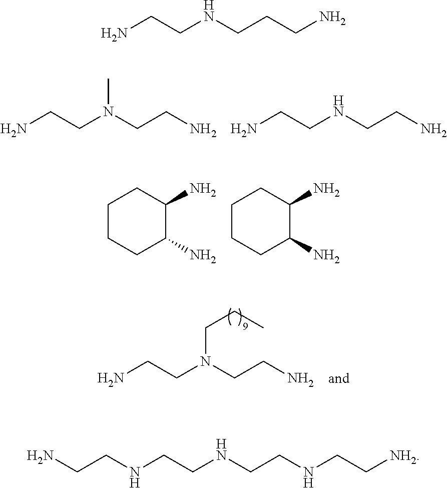

The term “alkyl” refers to a saturated, linear or branched hydrocarbon moiety, such as methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, and triacontyl.

The term “heteroalkyl” herein refers to an alkyl moiety containing at least one heteroatom selected from N, O, P, B, S, Si, Sb, Al, Sn, As, Se, and Ge.

Unless specified otherwise, both alkyl and heteroalkyl mentioned herein include substituted and unsubstituted moieties.

The antibody-conjugated nanoparticle contains a cytotoxin that is delivered into cells to exert a therapeutic effect. As pointed out above, the cytotoxin can be an unmodified natural protein or a natural protein modified with a chemical moiety.

In one embodiment, the cytotoxin contained in the nanoparticle is a natural protein modified with a chemical moiety. The chemical moiety generally contains an anionic group, a pH responsive group, a disulfide group, a hydrophobic group, a light responsive group, a reactive oxygen species responsive group, or a combination thereof. The chemical moiety can be linked to the natural protein via an amide group, an ester group, an ether group, a thioether group, a disulfide group, a hydrazone group, a sulfenate ester group, an amidine group, a urea group, a carbamate group, an imidoester group, or a carbonate group.

A natural protein contains a number of lysine residues, which show an electro-positive nature in physiological conditions. When a protein is modified with a chemical moiety containing an anionic group, e.g., carboxylate, by using an acid anhydride or a carboxylic acid-containing reagent, such chemical modification converts the positive lysine residues into negative carboxylates, thereby increasing the negative charge density of the protein. As a result, this modification improves loading of the protein into the antibody-conjugated nanoparticle via a strong electrostatic interaction between the modified anionic protein and the cationic lipid.

It should be pointed out that the protein modification is preferably reversible. Namely, after the modified protein, as a component of the nanoparticle, enters a cell, the chemical moiety can be cleaved as a result of pH change, or by a redox enzyme or light, to release the natural protein.

For example, a protein is modified with a pH responsive moiety by using an acid anhydride. After the modified protein enters a cell, the moiety is cleaved in a particular part of the cell, e.g., an endosome, due to the acidic condition, i.e., a pH value of 5-6. See Scheme 1(A) below. Note that the reversibility of the protein modification depends on the acid anhydride used. More specifically, as shown in Scheme 1(A), protein RNase A modified with cis-aconitic anhydride (a) and dimethylmaleic anhydride (b) are acid-labile but the one modified with succinic anhydride (c) is not. In this example, the pH responsive moiety contains an anionic group, i.e., carboxylate. The resulting protein is bonded to a cationic lipid for forming a nanocomplex via an electrostatic interaction.

As another example, a protein containing a lysine residue is modified with a disulfide moiety. After the modified protein enters a cell, the disulfide moiety is removed by glutathione (“GSH”) or other cysteine residues to regenerate the nascent protein. See Scheme 1(B) above. In this example, the disulfide moiety contains a hydrophobic alkyl group. The resulting protein is bonded to a cationic lipid for forming a nanocomplex via a hydrophobic interaction.

More examples of protein modification are described in Lee et al., Angewandte Chemie International Edition, 48, 5309-12 (2009); Lee et al., Angewandte Chemie International Edition, 49, 2552-55 (2010); and Maier et al., Journal of American Chemical Society, 134, 10169-73 (2012).

The lipid-based delivery agent and the cytotoxin described above include the compounds themselves, as well as their salts and solvates, if applicable. A salt, for example, can be formed between an anion and a positively charged group (e g, amino) on these compounds. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on these compounds. Suitable cations include sodium ion, potassium ion, magnesium ion, calcium ion, and an ammonium cation such as tetramethylammonium ion. The compounds also include those salts containing quaternary nitrogen atoms. A solvate refers to a complex formed between a lipid-like compound and a pharmaceutically acceptable solvent. Examples of pharmaceutically acceptable solvents include water, ethanol, isopropanol, ethyl acetate, acetic acid, and ethanolamine.

Also within the scope of this invention is a method of preparing the antibody-conjugated nanoparticle described above. Again, the method includes the following four steps: (i) providing a synthetic lipid formed from an electrophile and a primary or secondary amine, the electrophile being an epoxide, an acrylate, or an acrylamide; (ii) mixing the synthetic lipid and a cytotoxin to form a nanocomplex; (iii) mixing the nanocomplex with a lipid material to obtain a lipid-modified nanocomplex; and (iv) conjugating the lipid-modified nanocomplex with AMHRII antibody to form an antibody-conjugated nanoparticle that contains the cytotoxin. Note that the lipid material can be a commercially available lipid containing a reactive functional group. An exemplary lipid material is 1,2-distearoyl-sn-glycero-3-phosphoethanolamine-N-(cyanur(polyethylene glycol)-2000) (ammonium salt) or DSPE-PEG₂₀₀₀-Cyanur.

The antibody-conjugated nanoparticle thus obtained has a particle size of 50 to 1000 nm (e.g., 50 to 500 nm, 50 to 300 nm, and 50 to 180 nm).

This invention further covers a pharmaceutical composition containing the antibody-conjugated nanoparticle described above and a pharmaceutically acceptable carrier. The pharmaceutical carrier is compatible with the antibody-conjugated nanoparticle and should not be deleterious to a subject to be treated.

Still within the scope of this invention is a method of using the antibody-conjugated nanoparticle thus prepared for treating an AMHRII-associated condition in a subject, the method including two steps, i.e., identifying a subject that has an AMHRII-associated condition and administering to the subject in need thereof an effective amount of an antibody-conjugated nanoparticle, in which the antibody-conjugated nanoparticle delivers the cytotoxin contained therein into cells expressing AMHRII, thereby killing the cells.

“An effective amount” herein refers to the amount of the antibody-conjugated nanoparticle that is required to confer a sterilizing or therapeutic effect on the treated subject, e.g., inhibition of cancer cells growth. Effective doses will vary, as recognized by those skilled in the art, depending on the types of medical uses (i.e., sterilization or treatment of a disease), route of administration, excipient usage, and the possibility of co-usage with other medical treatment.

The antibody-conjugated nanoparticle of this invention can be used in treating various AMHRII-associated conditions, such as prostate cancer, breast cancer, endometrial cancer, cervical cancer, ovarian cancer, polycystic ovarian disease, and menopause.

A protocol of using nanoparticles for treating cancer is described in Wang et al., Angew. Chem., 126, 2937-2942 (2014).

To practice the method of the present invention, a composition having the above-described antibody-conjugated nanoparticle can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or di-glycerides). Fatty acid, such as oleic acid and its glyceride derivatives, are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition containing the antibody-conjugated nanoparticle can also be administered in the form of suppositories for rectal administration.

The antibody-conjugated nanoparticle covered by this invention can also be used in reproductive research such as surgical ovariectomies and/or castrations performed in a subject.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. The publications cited herein are incorporated by reference in their entirety.

Unless noted otherwise, all chemicals for lipidoid (i.e., a cationic lipid-like material) synthesis and protein modification were purchased from Sigma-Aldrich or Alfa-Aesar and used directly. Bovine pancreatic ribonuclease A (RNase A), saporin (from Saponaria officinalis), and anti-Mullerian hormone receptor II (AMHRII) antibody were purchased from Sigma-Aldrich. Commercial lipids used for in vivo injection formulations (1,2-dioleoyl-snglycero-3-phosphoethanolamine or DOPE, and DSPE-PEG₂₀₀₀/DSPE-PEG₂₀₀₀-Cyanur) were obtained from Avanti Polar Lipid, Inc.

### Example 1: Preparation of AMHRII Antibody-Conjugated Nanoparticles

Two AMHRII antibody-conjugated nanoparticles were prepared and characterized following the procedure described below.

A lipidoid (“EC16-1”) was prepared according to the method reported in Sun et al., Bioconjugate Chem., 2012, 23, 135-140.

A nanocomplex formed from EC16-1 and a protein, RNase A or saporin, was obtained by using a thin film hydration method reported in Wang et al., Angew. Chem., 2014, 126, 2937-2942. Briefly, EC16-1, cholesterol, and DOPE were mixed at a weight ratio of 16:2:1 in chloroform, and the organic solvent was then evaporated under vacuum to form a thin layer film. The thin layer film thus obtained was re-hydrated with phosphate-buffered saline, followed by addition of RNase A or saporin at a weight/weight ratio of 8:1 (EC16-1: protein) and incubation for 30 minutes at room temperature to afford an EC16-1/protein nanocomplex.

The EC16-1/protein nanocomplex was post-modified with DSPE-PEG₂₀₀₀/DSPE-PEG₂₀₀₀-Cyanur (weight/weight=3:1, 33% weight percentage in total compared to lipidoid) to provide a lipid-modified EC16-1/protein nanocomplex. 350 uL of the lipid-modified nanocomplex was then incubated with 20 μg of AMHRII antibody at 4° C. for 24 hours to afford an AMHRII antibody-conjugated nanoparticle.

The antibody-conjugated nanoparticles thus prepared, either using RNase A or saporin, had sizes of about 120-200 nm, determined by DLS analysis (Brookhaven ZetaPALS, NY) or TEM imaging (FEI, OR).

### Example 2: Use of an AMHRII Antibody-Conjugated Nanoparticle for Inducing Apoptosis

An AMHRII antibody-conjugated nanoparticle prepared in EXAMPLE 1 (saporin as the protein) was used for inducing apoptosis of gonad cells in rats following the procedure described below.

Male and female Sprague-Dawley rats were injected intravenously with saline, 2 nmol, 5 nmol, or 10 nmol of the AMHRII antibody-conjugated nanoparticle, or injected with 10 nmol of the nanoparticle directly into the gonads, i.e., testes or ovaries. The gonads were collected and flash frozen at 24 hours post injection. They were then cryostat-sectioned at −20° C., and 20 μm sections were thaw-mounted directly onto charged microscope slides. A terminal deoxynucleotidyl transferase dUTP nick end labeling (TUNEL) kit was used to detect DNA fragmentation as a result of apoptotic signaling cascades (In Situ Cell Death Detection Kit, TMR Red, Roche Applied Science, Indianapolis, Ind.) Immediately following the TUNEL assay, fluorescent immunohistochemistry was performed on the sections using an anti-caspase 8 primary antibody (Fisher Scientific, Hanover Park, Ill.) and alexa-fluor 488 as a secondary antibody (Life Technologies, Grand Island, N.Y.). The slides were cover-slipped with the Prolong Gold Anti-fade reagent with DAPI (Life Technologies, Grand Island, N.Y.) and allowed to cure overnight at room temperature prior to image production. Fluorescent images were obtained with a Zeiss Axiovert 200M fluorescent microscope (Carl Zeiss Microscopy, LLC, Thornwood, N.Y.) and quantification of tissue apoptosis was performed using ImageJ software (NIH, Bethesda, Md.). Data was analyzed with GraphPad Prism (La Jolla, Calif.) using two-way analysis of variance (ANOVA) to compare the number of TUNEL labeled apoptotic cells with sex and dose as the condition factors. Post hoc analyses were conducted using Tukey's test and significance was set to p<0.05.

Unexpectedly, at 24 hours post injection, significant apoptosis of granulosa and theca cells was observed for the ovaries of the female rats treated with the AMHRII antibody-conjugated nanoparticle, as compared to the female rats treated with saline; and significant apoptosis of sertoli and theca cells was observed for the testes of the male rats treated with the AMHRII antibody-conjugated nanoparticle, as compared to the male rats treated with saline.

Also unexpectedly, there was a main effect of sex (F_{[1,84]}=446.2, p<0.01), with males displaying more apoptotic cells than females. In addition, there was a main effect of dose (F_{[3,84]}=7.669, p<0.01) and an interaction between sex and dose (F_{[3,84]}=6.545, p<0.01). Male rats injected intravenously with 2 nmol of the nanoparticle showed less TUNEL labeling than all the other males. Female rats directly injected into the ovaries showed more TUNEL labeling than all the other females. Rats injected with saline showed negligible TUNEL signal.

These data indicate that the AMHRII antibody-conjugated nanoparticle of this invention is capable of delivering a cytotoxin into gonad cells and causing these cells to undergo apoptosis, thereby inducing sterilization in rats.

### Example 3: Use of an AMHRII Antibody-Conjugated Nanoparticle for Inducing Sterilization in Rats

An AMHRII antibody-conjugated nanoparticle prepared in EXAMPLE 1 (saporin as the protein) was used for inducing sterilization in female and male rats following the procedures described below.

Eight male and eight female Sprague Dawley rats (7 weeks of age), purchased from Charles River Laboratories (Shrewsbury, Mass., USA), were housed as same-sex pairs in shoe-box type cages with corncob bedding and Enviro-Dri® (Biological Associates, Melbourne, Australia) nest material for enrichment. Access to food (2018 Teklad global 18% protein rodent diet, Envigo, Cambridge, UK) and water was ad libitum. The rats were exposed to a 12:12 hour lighting schedule with lights on during working hours. All animal work was approved by Tufts Institutional Animal Care and Use Committee (IACUC) and performed under IACUC Protocol G2015-158.

The rats were divided into two groups: one group injected with the AMHRII antibody-conjugated nanoparticle (rats N1-N8) and the other group injected with sterile saline (rats S1-S8). More specifically, the N1-N8 rats received 8.2 nmol intravenous dose of the nanoparticle in 0.1 ml of sterile saline followed by another 0.1 mL of saline using a 24 g over-the-needle-catheter inserted into the lateral tail vein, the additional 0.1 mL of saline being used as a flush to ensure that all nanoparticle was fully delivered; and the S1-S8 rats received two 0.1 mL doses of sterile saline in the same manner as the N1-N8 rats.

All rats, injected on Day 0, were housed for 4 weeks and weighed twice a week on Mondays and Thursdays at approximately 12-2 pm. Females were assessed for estrous cyclicity during Weeks 3 and 4. The rats were sacrificed by exposure to CO2. Blood was collected, testes and uteri were weighed, epididymides were collected for semen evaluation, and ovaries and testes were preserved in 10% formaldehyde for histological processing.

The AMHRII antibody-conjugated nanoparticle did not produce any significant side effects. More specifically, it was observed that, for both the weight of testes and the weight of uterus (without ovaries), there was no appreciable difference between the rats treated with the AMHRII antibody-conjugated nanoparticle and those treated with saline. Also, no adrenal gland apoptosis was observed in all rats.

### Evaluation of Female Vaginal Smears

Starting Week 3, the female rats were checked daily (between 9-11 am) for estrous cyclicity by performing vaginal lavages and evaluating vaginal cytology as follows.

Rats were manually restrained and a small pipette containing 0.25 mL (maximum volume) of sterile saline was inserted into the caudal vagina. The saline was injected into the vagina and immediately aspirated. The aspirate was examined using an Olympus BX50 microscope (Olympus Corporation of the Americas Headquarters, Center Valley, Pa., USA) fitted with a Photometrics CoolSNAP HQ2 video camera (Photometrics, Tucson, Ariz., USA) to determine the stage of the estrous cycle by identifying the types of cells in the aspirate. Pictures at 100×, 200×, and 400× were taken for every aspirate using MetaMorph® (Nashville, Tenn., USA) image analysis software.

Shown in Table 1 below are the female vaginal smear data from the rats treated with the AMHRII antibody-conjugated nanoparticle or saline.

TABLE 1
Female vaginal smear data
Female Vaginal Smear

| | Day 22 | Day 23 | Day 24 | Day 25 | Day 26 | Day 27 | Day 28 | |
|---|---|---|---|---|---|---|---|---|
| N5 | D | D/A | E/A | M/A | M/A | P/A | E/A | Acyclic |
| N6 | E/A | E/A | M/A | M/A | M-D/A | M/A | P-E/A | Acyclic |
| N7 | M/A | M/A | E/A | M/A | D/A | M | P-E/A | Acyclic |
| N8 | P-E/A | P/A | E | M/A | D | E-M | P-E/A | Acyclic |
| S5 | P | E | D | D | P | D sparse | M-D | Cycling |
| S6 | D | D | D | D-P | D sparse | D | D sparse | Pseudo |
| S7 | E | M | D | D | D | P | E | Cycling |
| S8 | E | M | D | P | E | M | M-D | Cycling |

Notes:
(i) N5-N8 represent the rats treated with the AMHRII antibody-conjugated nanoparticle;
(ii) S5-S8 represent the rats treated with saline; and
(iii) A denotes all cell types, D denotes diestrus, E denotes estrus, M denotes metoestrus, and P denotes proestrus.

Table 1 demonstrates that the groups of rats treated with the AMHRII antibody-conjugated nanoparticle, i.e., N5-N8, had smears with many cells and various cell types. As a result, it was difficult to determine any part of the cycle definitively. Note that the smears represent those observed in senescent rats and in rats induced to exhibit polycystic ovarian disease as a model. By contrast, the groups of rats treated with saline, i.e., S5-S8, had smears indicating normal cycling except S6 which exhibited diestrus smears consistent with pseudopregnancy. Clearly, the tested rats treated with the AMHRII antibody-conjugated nanoparticle demonstrated acyclicity, an indicium of sterilization, as compared to those treated with saline.

### Histological Evaluation of Ovaries

Gonadal tissue embedded with formalin-fixed paraffin was sliced from the central axis of each ovary at 5 μm per section. Sections were mounted on slides, alternating sections among 3 slides. One slide was stained with hematoxylin and eosin to evaluate gonadal architecture. Another slide was used to identify specific cells undergoing apoptosis using TUNEL. A third slide was produced to serve as a backup that could eventually be used to identify AMHII receptors using a fluorescent antibody technique. Tissue preparation and staining was performed by the Histopathology Section at the Cummings Veterinary School. Certain Images were captured using a Zeiss Axiovert 200M fluorescent microscope (Carl Zeiss Microscopy) outfitted with a Zeiss AxioCam MRm camera and Zen software.

Shown in Table 2 below are the number of corpora lutea and follicles from left and right ovaries of rats treated with saline or the AMHRII antibody-conjugated nanoparticle.

TABLE 2
Number of cell types in combined left and right ovaries

| | CL | Pre-ov | Tertiary | Secondary | Total |
|---|---|---|---|---|---|
| Animal | | | | | |
| S5 | 19 | 2 | 18 | 16 | 55 |
| S6 | 10 | 9 | 10 | 9 | 38 |
| S7 | 11 | 0 | 10 | 15 | 36 |
| S8 | 15 | 4 | 12 | 13 | 44 |
| Average | 13.75 | 3.75 | 12.5 | 13.25 | 43.25 |
| (S5-S8) | | | | | |
| N5 | 22 | 1 | 28 | 12 | 63 |
| N6 | 20 | 0 | 15 | 7 | 42 |
| N7 | 30 | 0 | 26 | 18 | 74 |
| N8 | 18 | 0 | 28 | 14 | 60 |
| Average | 22.5 | 0.25 | 24.25 | 12.75 | 59.75 |
| (N5-N8) | | | | | |
| T-test | 0.039538 | 0.122387 | 0.018128 | 0.862273 | 0.081532 |
| (2 tails) | | | | | |
| | P < 0.05 | | P < 0.05 | | |

Notes:
(i) S5-S8 represent the rats treated with saline;
(ii) N5-N8 represent the rats treated with the AMHRII antibody-conjugated nanoparticle; and
(iii) CL denotes corpus luteum; Pre-ov denotes preovulatory follicle; Tertiary denotes tertiary/antral follicle, and Secondary denotes secondary follicle.

As shown in Table 2, the group of rats treated with the AMHRII antibody-conjugated nanoparticle, i.e., N1-N4, had a significantly higher (P<0.05) number of tertiary follicles, i.e., an average number of 24.25, as compared to an average number of 12.5 exhibited by those treated with saline, i.e., S5-S8. Further, the N1-N4 rats had a significantly higher (P<0.05) number of corpora lutea and fewer pre-ovulatory follicles, i.e., respective average numbers of 22.5 and 0.25, as compared to those exhibited by the S5-S8 rats, i.e., respective average numbers of 13.75 and 3.75. Of note, an abundance of tertiary follicles combined with a large number of corpora lutea and few pre-ovulatory follicles are seen in female rat models of Polycystic Ovatrian Syndrome and senescence where fertility is impaired or eliminated.

These data indicate that the AMHRII antibody-conjugated nanoparticle of this invention is capable of inducing sterilization in female rats.

### Evaluation of Male Epididymal Sperm

For each of the tested rats, the tail of the epididymis was removed along with approximately ¼″ of ductus deferens and placed in a small micro centrifuge tube containing normal saline. The samples were stood at room temperature for about 6 hours. At the time of examination, 100 μL of fluid was aspirated from each tube and a drop (about 50 μL) was placed on a slide. All samples were examined using an Olympus BX50 microscope (Olympus Corporation of the Americas Headquarters, Center Valley, Pa., USA) fitted with a Photometrics CoolSNAP HQ2 video camera (Photometrics, Tucson, Ariz., USA). Pictures at 200× were taken of every aspirate using MetaMorph® (Nashville, Tenn., USA) image analysis software

The test results are shown in Table 3 below.

TABLE 3
Male epididymal sperm data:
Male Epididymal Sperm

| Male | N1 | N2 | N3 | N4 | S1 | S2 | S3 | S4 |
|---|---|---|---|---|---|---|---|---|
| Left | X | XX | X | XX | XXX | XXX | XXX | XX |
| testis | D | LL | L | L | LL | LL | LL | L |
| Right | X | X | X | X | XXX | XX | XXX | XXX |
| testis | L | D | L | L | LL | LL | LL | LL |

Notes:
(i) N1-N4 represent the rats treated with the AMHRII antibody-conjugated nanoparticle;
(ii) S1-S4 represent the rats treated with saline; and
(iii) X denotes few sperms, XX denotes a moderate number of sperms, XXX denotes a large number of sperms, L denotes less than 50% of sperms alive, LL denotes 50% or greater of sperms alive, and D denotes all sperms dead.

As shown in Table 3, the groups of rats treated with the AMHRII antibody-conjugated nanoparticle, i.e., N1-N4, had far fewer sperms in both testes than those treated with saline, i.e., S1-S4. In addition, the N1-N4 groups of rats had fewer live (i.e., moving) sperms than the S1-S4 groups, resulting from apoptosis of the sertoli cells in the testes induced by the AMHRII antibody-conjugated nanoparticle.

These data indicate that the AMHRII antibody-conjugated nanoparticle of this invention is capable of inducing sterilization in male rats.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. An antibody-conjugated nanoparticle comprising:
an anti-Mullerian hormone receptor II (AMHRII) antibody, and
a nanocomplex formed of a lipid-based delivery agent and a cytotoxin, the delivery agent and the cytotoxin being non-covalently bonded to each other,
wherein the AMHRII antibody is conjugated to the nanocomplex to form a nanoparticle that contains the cytotoxin, the nanoparticle having a size of 50 to 1000 nm.

2. The antibody-conjugated nanoparticle of claim 1, wherein the lipid-based delivery agent contains a cationic lipid.

3. The antibody-conjugated nanoparticle of claim 2, wherein the cytotoxin is an unmodified natural protein or a natural protein modified with a chemical moiety.

4. The antibody-conjugated nanoparticle of claim 3, wherein the cytotoxin is a natural protein modified with a chemical moiety.

5. The antibody-conjugated nanoparticle of claim 3, wherein the cytotoxin is RNase A-Aco, saporin, or saporin-Aco.

6. The antibody-conjugated nanoparticle of claim 4, wherein the chemical moiety contains an anionic group, a pH responsive group, a disulfide group, a hydrophobic group, a light responsive group, a reactive oxygen species responsive group, or a combination thereof.

7. The antibody-conjugated nanoparticle of claim 4, wherein the chemical moiety is linked to the natural protein via an amide group, an ester group, an ether group, a thioether group, a disulfide group, a hydrazone group, a sulfenate ester group, an amidine group, a urea group, a carbamate group, an imidoester group, or a carbonate group.

8. The antibody-conjugated nanoparticle of claim 7, wherein the chemical moiety is linked to the natural protein via an amide group, an ester group, a disulfide group, a thioester group, or a carbamate group.

9. The antibody-conjugated nanoparticle of claim 8, wherein the chemical moiety contains an anionic group, a pH responsive group, or a disulfide group.

10. The antibody-conjugated nanoparticle of claim 2, wherein the cytotoxin is a small molecule having a molecular weight of 900 Daltons or less.

11. The antibody-conjugated nanoparticle of claim 10, wherein the cytotoxin is pacilitaxel or doxorubicin.

12. The antibody-conjugated nanoparticle of claim 2, wherein the cationic lipid is formed from a primary or secondary amine and an electrophile selected from the group consisting of an epoxide, an acrylate, and an acrylamide.

13. The antibody-conjugated nanoparticle of claim 12, wherein the cationic lipid is formed from a primary or secondary amine and an epoxide, in which the epoxide is 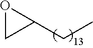
and the primary or secondary amine is selected from the group consisting of

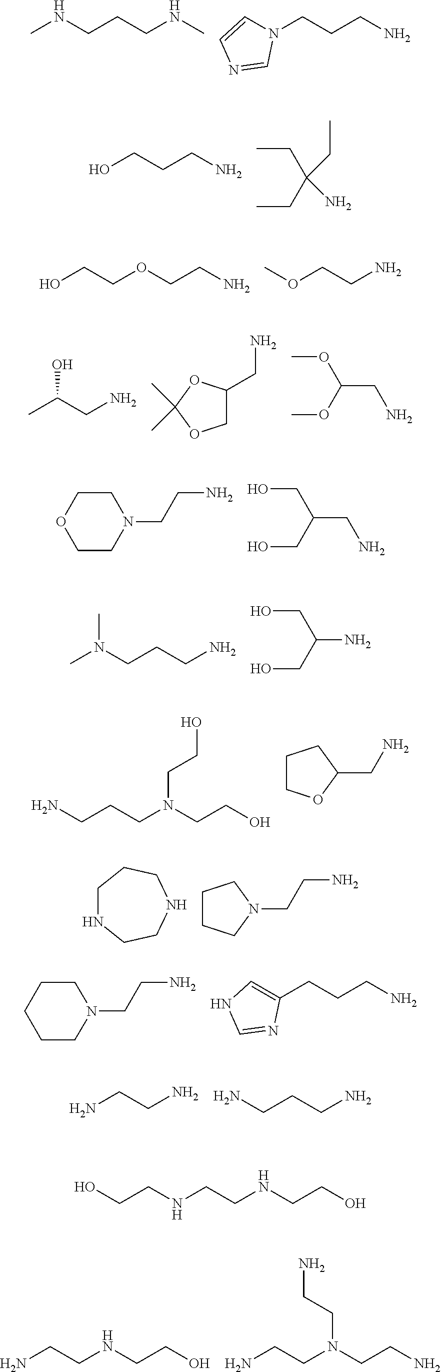

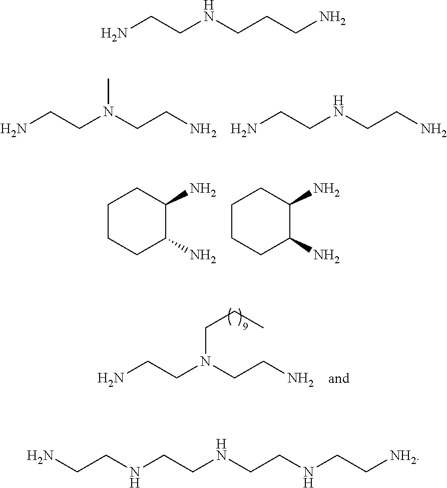

14. The antibody-conjugated nanoparticle of claim 2, wherein the cationic lipid contains a disulfide bond and is bioreducible.

15. The antibody-conjugated nanoparticle of claim 2, wherein the cationic lipid is formed from a primary or secondary amine and an epoxide and the cytotoxin is RNase A-Aco, saporin, or saporin-Aco.

16. The antibody-conjugated nanoparticle of claim 15, wherein the epoxide is 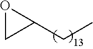
and the primary or secondary amine is selected from the group consisting of

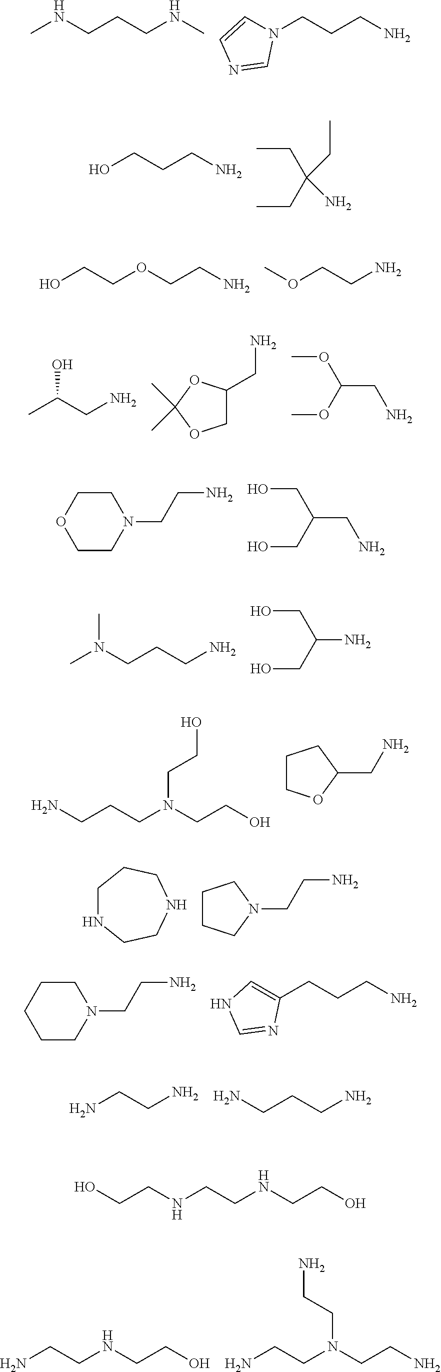

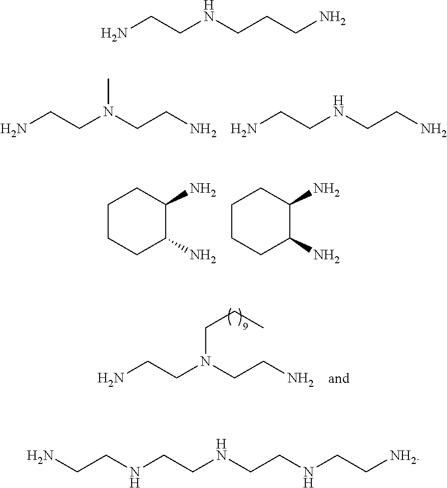

17. The antibody-conjugated nanoparticle of claim 1, wherein the lipid-based delivery agent is bonded to the cytotoxin via an electrostatic interaction or a hydrophobic interaction.

18. A method of preparing an antibody-conjugated nanoparticle of claim 1, the method comprising:
providing a synthetic lipid formed from an electrophile and a primary or secondary amine, the electrophile being an epoxide, an acrylate, or an acrylamide;
mixing the synthetic lipid and a cytotoxin to form a nanocomplex;
mixing the nanocomplex with a lipid material to obtain a lipid-modified nanocomplex; and
conjugating the lipid-modified nanocomplex with AIMIHRII antibody to form an antibody-conjugated nanoparticle that contains the cytotoxin.

19. A method of inducing sterilization in a subject, the method comprising:
identifying a subject in need of sterilization, and
administering to the subject an effective amount of an antibody-conjugated nanoparticle of claim 1,
whereby the antibody-conjugated nanoparticle delivers the cytotoxin contained therein into gonad cells and suppresses the formation of sperm or ova, thereby inducing sterilization in the subject.

20. The method of claim 19, wherein the antibody-conjugated nanoparticle contains a nanocomplex formed from a cationic lipid and a cytotoxin, in which the cationic lipid is prepared by reacting an epoxide with a primary or secondary amine and the cytotoxin is RNase A-Aco, saporin, or saporin-Aco.

21. The method of claim 20, wherein the epoxide is 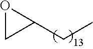
and the primary or secondary amine is selected from the group consisting of

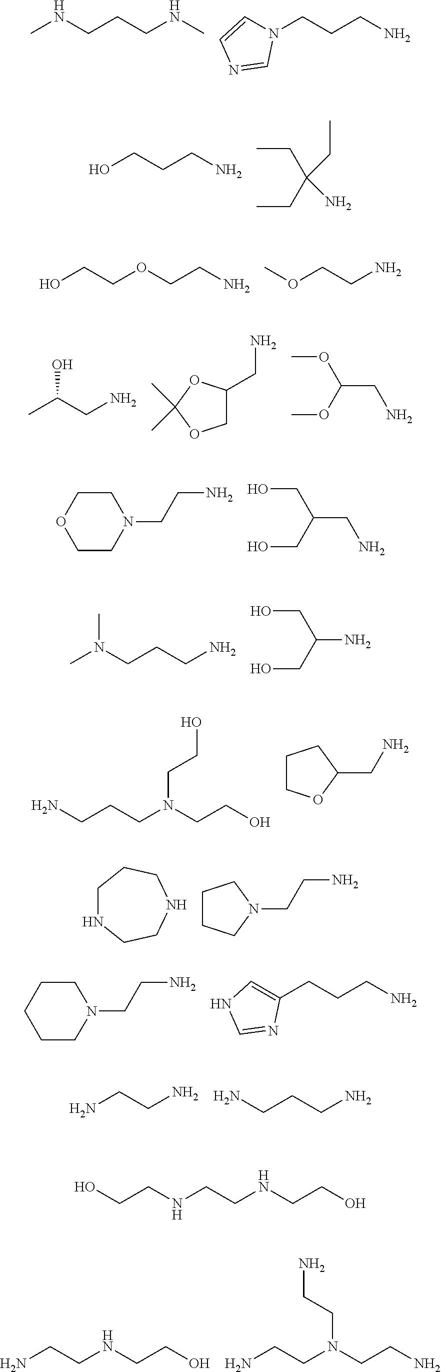

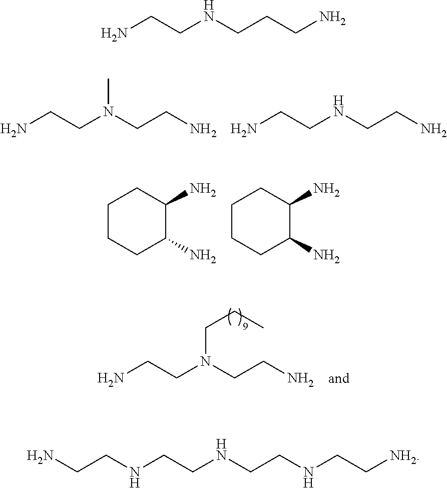

22. A method of treating an AMHRII-associated condition in a subject, the method comprising:
identifying a subject that has an AMHRII-associated condition, and
administering to the subject in need thereof an effective amount of an antibody-conjugated nanoparticle of claim 1,
whereby the antibody-conjugated nanoparticle delivers the cytotoxin contained therein into cells expressing AMHRII, thereby killing the cells.

23. The method of claim 22, wherein the AMHRII-associated condition is selected from the group consisting of prostate cancer, breast cancer, endometrial cancer, cervical cancer, ovarian cancer, polycystic ovarian disease, and menopause.

24. (canceled)

25. The method of claim 22, wherein the antibody-conjugated nanoparticle contains a nanocomplex formed from a cationic lipid and a cytotoxin, in which the cationic lipid is prepared by reacting an epoxide with a primary or secondary amine and the cytotoxin is RNase A-Aco, saporin, or saporin-Aco.

26. The method of claim 25, wherein the epoxide is 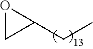
and the primary or secondary amine is selected from the group consisting of

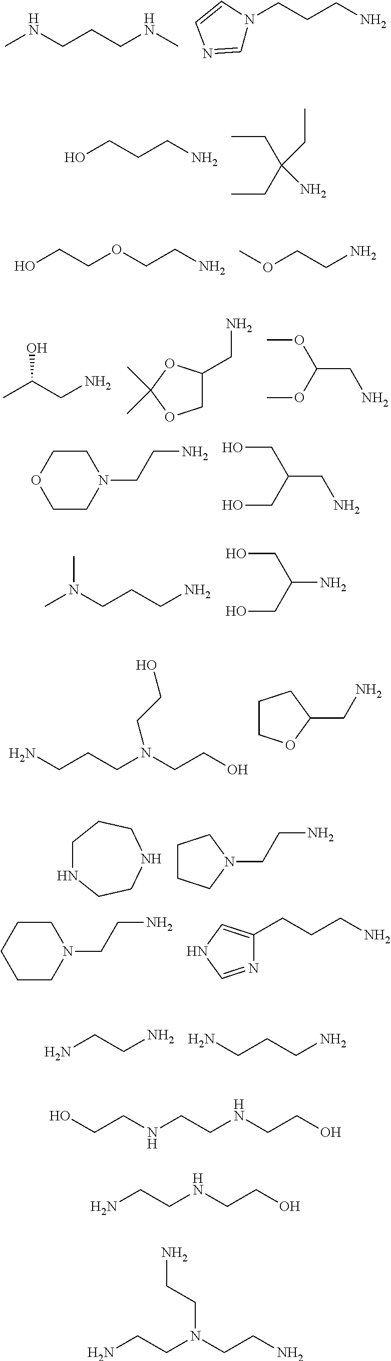

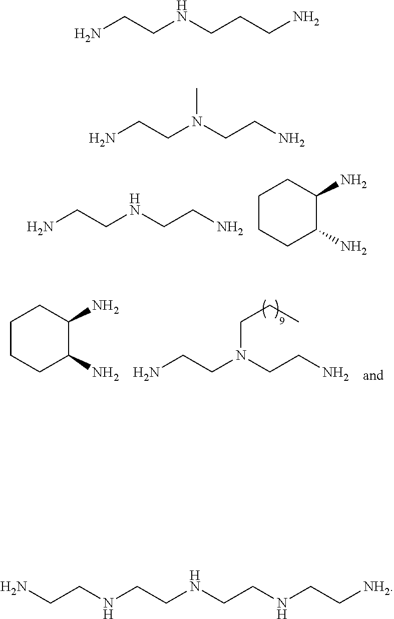

27. (canceled)
